(19) United States
(12) Patent Application Publication
(10) Pub. No.: US 2021/0236494 A1
(43) Pub. Date: Aug. 5, 2021

(54) THERAPEUTIC COMBINATION

(71) Applicant: Oncologica UK Ltd., Chesterford, Cambridge (GB)

(72) Inventors: Gareth Williams, Ely, Cambridgeshire (GB); Marco Loddo, Saffron Walden, Essex (GB)

(21) Appl. No.: 17/052,006

(22) PCT Filed: May 1, 2019
(86) PCT No.: PCT/GB2019/051205

§ 371 Date: Oct. 30, 2020

(30) Foreign Application Priority Data
May 1, 2018 (GB) 1807147.2

(51) Int. Cl. A61K 31/513; A61K 31/337; A61K 45/06

(52) U.S. Cl. CPC A61K 31/513; A61K 45/06; A61K 31/337

(57) ABSTRACT

There is provided a combination of agents which allow normal cells to be reversibly arrested in G1 phase thereby shielding them from the toxic effects of cytotoxic agents used to kill cancer cells in cancer treatment.

The present invention relates to combinations of agents which are useful in the protection of normal cells during cancer treatment with a cytotoxic agent, to kits of pharmaceutical compositions comprising these, and methods of treatment and dosage regimes which utilise these combinations.

### BACKGROUND OF THE INVENTION

The presence of an origin activation checkpoint which arrests cells in G1 in response to perturbations in DNA replication initiation is supported by experimental evidence from several studies. This checkpoint in the DNA licensing machinery or DNA replication initiation machinery can be induced using many mechanisms including RNAi against Cdc7, an essential kinase involved in the initiation of DNA synthesis at licensed chromosomal replication origins though phosphorylation and activation of the Mcm2-7 helicase. Other targets for disruption of the checkpoint have been found to be ORC1-6, Cdc6, Cdt1, geminin, Dbf4 Cdc45, GINS, Polε, Mcm10, Sid3, Sid5, Sid7, Sid2, Dpb11, Polα, Ctf4, PCNA, Pfs1, Pfs2 and Psf3.

In normal cells, the checkpoint prevents entry into a lethal S phase in the presence of an insufficient number of replication competent origins. In contrast, many cancer cells have a defective checkpoint, which leads to fork stalling/collapse, an abortive S phase and apoptotic cell death.

The molecular architecture of the origin activation checkpoint has recently been characterised and it has been shown that in normal cells arrest of cells in G1 phase can be reversed. (Rodriguez-Acebes S, et al. Am J Pathol 2010; 177:2034-45; PMID: 20724597; DOI:10.2353/ajpath.2010.100421; Tudzarova S, et al. EMBO J 2010; 29:3381-94; PMID: 20729811; DOI: 10.1038/emboj.2010.201). The checkpoint response in arrested cells was shown to be dependent on 3 non-redundant axes mediated by FoxO3a, involving upregulation of CDK inhibitor p15INK4B, activation of the p14ARF_MDM2_p53_p21 pathway, and p53 mediated upregulation of the Wnt/_-catenin pathway antagonist DKK3, which leads to Myc and cyclin D1 downregulation. The resulting loss of CDK activity inactivates the Rb-E2F pathway, overrides the G1-S transcriptional program and leads to a robust G1 cell cycle arrest.

The involvement of several tumour suppressor genes (TSGs) frequently inactivated during tumorigenesis and the lack of redundancy may account for why cancer cells have a defective origin activation checkpoint arrest. Furthermore, this provides a mechanistic basis for the cancer-cell-specific killing observed with emerging pharmacological Cdc7 inhibitors (Montagnoli A. et al., Nat Chem Biol 2008; 4:357-65; PMID: 18469809; DOI: 10.1038/nchembio).

Anti-mitotic chemotherapeutic agents remain a cornerstone of multimodality treatment for locally advanced and metastatic cancers. For example, the potent anti-mitotic taxane, paclitaxel, is broadly used in neoadjuvant/adjuvant therapy and also in the treatment of metastatic disease. A drawback of these current chemotherapy regimens, however, remains the associated toxicity in normal tissues with high cellular turnover, for example of the bone marrow, hair follicle cells, and gastrointestinal tract epithelium. This often leads to undesired side effects such as myelosuppression (e.g. neutropenia), hair loss and gastrointestinal toxicity, and consequently dose reduction and incomplete administration of prescribed regimens, allowing survival of tumor cells and the development of drug resistance.

Therefore novel approaches to enhance the therapeutic window of existing cytotoxic chemotherapies are required.

Cyclotherapy is a strategy aimed at exploiting differences between normal and cancer cells to selectively protect normal proliferating cells from the cytotoxic effects of chemotherapy, thereby increasing the therapeutic window (Blagosklonny M V et al. Cell Cycle 2001, 1:375-82: PMID: 12548008; DOI: 10.4161/cc.1.6.259; Blagosklonny M V et al. Cancer Research 2001, 4301-4305). This is based on the concept that as most cytotoxic chemotherapies preferentially target cycling cells, by selectively inducing a reversible cell cycle arrest in normal cells, these cells would thus be protected from cytotoxicity, and can re-enter the cell cycle unharmed. In contrast, cancer cells which are characterized by uncontrolled proliferation as the result of multiple genetic aberrations and loss of checkpoint regulation fail to cell cycle arrest and remain sensitive to cytotoxic chemotherapy. In summary, triggering the DNA replication initiation checkpoint in normal cells induces a reversible G1 arrest which therefore protects these cells from S phase and G2/M phase directed chemotherapeutic agents.

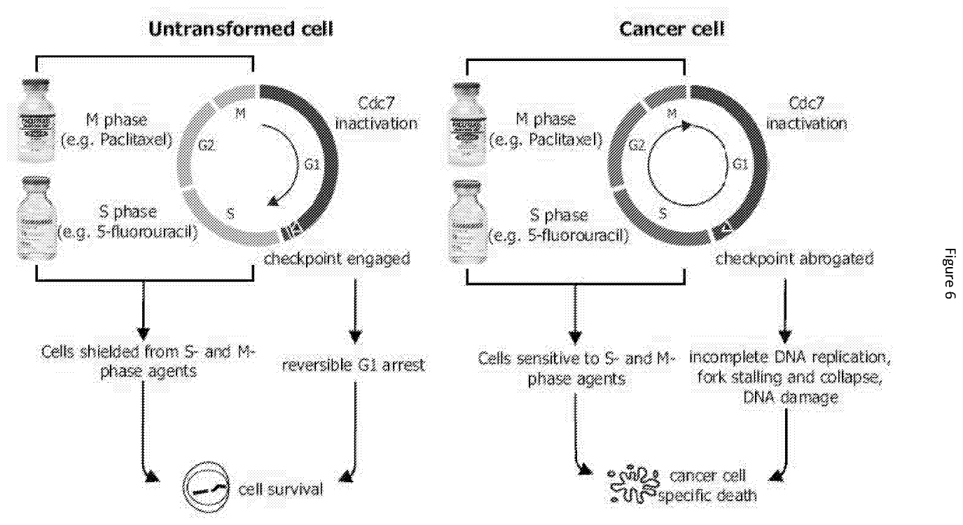

Many components of the DNA replication machinery/pathway have been proposed as anti-cancer drug targets. Cdc7 kinase in particular has been identified as an important target. Inhibition of DNA origin firing by targeting Cdc7 kinase with ATP-competitive SMIs or RNAi results in cancer cells entering an abortive S phase followed by apoptotic cell death. It has been shown (Mulvey et al. Journal of Proteome Research 2010, 9, 5445-5460) that normal somatic cells avoid entering a lethal S phase by engaging a DNA origin activation checkpoint that reversibly arrests cells in G1 phase, as illustrated schematically in FIG. 6. As a result, therapies involving such inhibitors would provide a specific and selective anti-tumour effect, which left normal cells undamaged, thus reducing unwanted side effects.

The pharmaceutical industry have selected in particular Cdc7 kinase and the MCMs as potential therapeutic targets and there are many drug development programmes in place throughout the world developing anti-cancer agents targeting these DNA replication licensing/initiation proteins. In general, this has focused on the generation of small molecule compounds but targeting of factors such as Cdc7 kinase could also be achieved using a range of biological agents such as RNAi, inhibitory peptides or immunoglobulins such as monoclonal antibodies or binding fragments thereof.

### SUMMARY OF THE INVENTION

According to the present invention there is provided a combination of i) an inhibition or disruption agent which inhibits or disrupts the DNA licensing machinery and/or the DNA replication initiation machinery and ii) a cytotoxic agent which acts in either the G2, M and/or S phases of a cell cycle for use in the shielding of normal cells during cancer treatment, wherein the inhibition or disruption agent is administered to the patient first in an amount sufficient to reversibly arrest normal cells in G1 phase, and the cytotoxic agent ii) is administered subsequently.

The inhibition or disruption agent which inhibits or disrupts the DNA licensing machinery and/or the DNA replication initiation machinery can be any suitable agent which acts at the protein or nucleotide level to inhibit or disrupt the entry of the normal cell into G2, M or S phases. The normal cell is, therefore, arrested within the G1 phase.

Using such an agent ensures that the normal cells are shielded or protected from the cytotoxic agents used to kill the cancer cells as the cancer cells remain actively replicating and so are susceptible to the toxic effects of the cytotoxic agent.

The inhibition or disruption agent which inhibits or disrupts the DNA licensing machinery and/or the DNA replication initiation machinery may be administered at levels lower than that necessary to produce a cytotoxic effect, and therefore unwanted side effects from this agent may be reduced.

Preferably the inhibition or disruption agent inhibits or disrupts one or more of Cdc-7, ORC1-6, Cdc6, MCM2-7, Cdt1, geminin, Dbf4 Cdc45, GINS, Polε, Mcm10, Sid3, Sid5, Sid7, Sid2, Dpb11, Polα, Ctf4, PCNA, Pfs1, Pfs2 and Psf3.

Conveniently the inhibition or disruption agent is selected from a suitable RNAi drug, a small molecule inhibitor, a peptide or a therapeutic monoclonal antibody. Such agents are readily available and a person skilled in the art would be able to identify the same.

Conveniently, there is administered only one inhibition or disruption agent.

Preferably, the inhibition or disruption agent is directed towards Cdc7. It should be noted that the term Cdc7 or any other gene mentioned herein may also refer to the protein. If the protein is being specifically referred to the term ‘protein’ will be placed thereafter. Likewise, the term CDC7 or any other protein mentioned herein may also refers to the gene. If the gene is being specifically referred to the term ‘gene’ will be placed thereafter.

Suitable inhibitors may act at either the protein level, for example by binding to the CDC7 protein to inactivate it, or it may act at the nucleotide level, so that gene expression or translation is downregulated.

Examples of inhibitors that act at the protein level may comprise small molecules, in particular small competitor molecules, aptamers, as well as antibodies or binding fragments thereof.

Suitable antibody binding fragments include Fab, Fab′, F(ab)2, F(ab′)2 and FV, VH and VK fragments. Antibodies may be monoclonal or polyclonal but in particular are monoclonal antibodies. Whilst the antibody may be from any source (murine, rabbit etc.), for human therapeutic use, they suitably comprise a human antibody or an antibody which has been partly or fully humanised.

Examples of inhibitors that act at the nucleotide level include transcription regulators that may prevent gene expression, or RNA inhibitors such as RNA molecules or nanomolecules that target the relevant DNA replication machinery and/or DNA licensing machinery, for example, Cdc7. These may include anti-sense RNA constructs an RNA molecule such as a small interfering RNA (siRNA), a short hairpin RNA (shRNA), a microRNA (miRNA), or a short activating RNA (saRNA) which are designed to silence or inactivate the relevant gene.

Suitable small molecule inhibitors which specifically target CDC7 include thiophenes, thiazolidinones, pyrrolopyridines, pyrrolopyazines, pyrrolopyrazinones pyrimidines, imidazolones, phthalazinones, furanones, azaindole or isoindolinones such as described for example in WO2005095386, WO2005014572, WO2007110344, US20090253679, US2011190299, WO2010101302, WO2011008830, WO2011008915, WO2011112635, WO2011130478, WO2011130481, WO201113388, US20120135989, US20140018533 or WO2014143601, the content of which is incorporated herein by reference. Particular examples include TAK-931, LY-3143921, LBS-007, SRA-141, NMS-1116354, BMS-863233, RXDX-103, MSK-777 and MSK-747. Suitable cytotoxic agents which may be used in the combination of the invention include any of the cytotoxins available for use in anti-cancer therapy, provided they act specifically in the G2, M or S phase of the cell cycle. Conveniently, one or more cytotoxic agents is administered depending upon the treatment prescribed by the physician. Preferably, one cytotoxic agent is administered.

Inhibitors and disrputors of DNA licensing machinery and/or the DNA replication initiation machinery will be well known to those in the art but will include SMoC-geminin as detailed in Nature Methods, vol. 4, pg 153-159 (2007) and RNAi against ORC2 as detailed in The EMBO Journal (2010) 29, 3381-3394.

Examples of S-phase agents may comprise antimetabolites (e.g. 5-Azacytadine, cytarabine, fludarabine, 5-flourouracil (5-FU), FUDR, gemcitabine, hydroxyurea, leustatin, 6-mercaptopurine, methotrexate, pentostatin, 6-thioguanine, cytosine, arabinoside, fluoxouridine, pemetrexed.

M-phase agents may include Plant alkaloids, etoposide, topoisomerase II, teniposide, vinblastine, Vincristine (VCR), vindesine, Vinorelbine or Taxanes (paclitaxel, docetaxel).

G2-phase agents include pleomycin, etoposide, topotecan, daunorubicin.

Other agents which have the potential to affect S, G2 or M phase include topoisomerase inhibitors, alkylating agents (eg. nitrogen mustards; ethylenimes; alkylsulfonates; triazenes; piperazines; and nitrosureas), an antimetabolite (eg mercaptopurine, thioguanine, 5-fluorouracil), a mitotic disrupter (eg. plant alkaloids-such as vincristine and/or microtubule antagonists-such as paclitaxel), a DNA synthesis inhibitor, a DNA-RNA transcription regulator, an enzyme inhibitor, a gene regulator, a hormone response modifier, or a combination of two or more thereof.

The chemotherapeutic agent may be an anti-metabolite such as methotrexate and 5-fluorouracil (5-FU); a taxoid such as paclitaxel (TAXOL®), abraxane, and/or TAXOTERE®, doxetaxel. Preferably the cytotoxic agent is a taxane. Conveniently the cytotoxic agent is selected from paclitaxel or docetaxel.

Since the cytotoxic agent (ii) used acts in either the G2, M or S phase of the cell cycle, this means that normal cells, which have been reversibly arrested in the G1 phase by the CDC7 inhibitor will be protected, as illustrated hereinafter in FIG. 6.

There are many known examples of chemotherapy compounds used as anti-cancer agents, which act in the S or G2 or M phase of the cell cycle. Particular examples for use in the present invention include paclitaxel and 5-fluorouracil.

Any form of cancer which uses a treatment that acts in the G2, M and/or S phases can be treated using this combination as the inhibition or disruption agent used to shield or protect the cancer stalls the normal cells reversibly within the G1 phase but does not have the same effect on the cancerous cells.

Preferably, cancers which may be treated by the combination of the invention include solid cancers such as Adrenal Cancer, Anal Cancer, Basal and Squamous Cell Skin Cancer, Bile Duct Cancer, Bladder Cancer, Bone Cancer, Brain and Spinal Cord Tumors, Breast Cancer, Cancer of Unknown Primary, Cervical Cancer, Colorectal Cancer, Endometrial Cancer, Esophagus Cancer, Ewing's sarcoma, Eye Cancer, Gallbladder Cancer, Gastrointestinal Carcinoid, Gastrointestinal Stromal Tumor (GIST), Kidney Cancer, Laryngeal and Hypopharyngeal Cancer, Liver Cancer, Lung Cancer, Lung, Carcinoid Tumor, Malignant Mesothelioma, Melanoma, Merkel Cell Skin Cancer, Nasal Cavity and Paranasal Sinuses Cancer, Nasopharyngeal Cancer, Neuroblastoma, Non-Small Cell Lung Cancer, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Skin Cancer, Small Cell Lung Cancer, Small Intestine Cancer, Soft Tissue Sarcoma, Stomach Cancer, Testicular Cancer, Thymus Cancer, Thyroid Cancer, Uterine Sarcoma, Vaginal Cancer, Vulvar Cancer as well as haematological malignancies, such as leukaemia and lymphomas including chronic myelogenous leukemia, chronic myelomonocytic leukemia, Philadelphia chromosome positive acute lymphoblastic leukemia and mantle cell lymphoma.

Conveniently, the cancers are selected from ovarian carcinoma, osteocarcinoma, breast carcinoma and small cell lung carcinoma.

For use in these therapies, the combination of the invention is suitably administered in the form of pharmaceutical compositions. The agents of the combination are administered individually and sequentially to allow agent (i) to arrest normal cells in the G1 phase, before they are exposed to the cytotoxic agent (ii). In order to achieve this, the CDC7 inhibitor (i) is administered first, before any cytotoxic agent has been administered to the patient. The time between administration of agent (i) and agent (ii) is suitably in the range of from 1-60 hours, for example from 12 to 48 hours, such as from 24 to 36 hours.

In such cases, the agents will suitably be in the form of individual pharmaceutical compositions, which may be packaged together, for instance in blister packs or the like and arranged in dosage units. Alternatively, the sequential administration could be a single composition which has been suitably formulated such that the different agents have different release profiles.

Suitable pharmaceutical compositions will be in either solid or liquid form. They may be adapted for administration by any convenient peripheral route, such as parenteral, oral, vaginal or topical administration or for administration by inhalation or insufflation. The pharmaceutical acceptable carrier may include diluents or excipients which are physiologically tolerable and compatible with the active ingredient. These include those described for example in Remington's Pharmaceutical Sciences.

The pharmaceutical compositions may have the same or different routes of administration and, therefore, the same or different forms (e.g tablet and chemo infusion)

Parenteral compositions are prepared for injection, for example subcutaneous, intramuscular, intradermal, and intravenous or via needle-free injection systems. Also, they may be administered by intraperitoneal injection. They may be liquid solutions or suspensions, or they may be in the form of a solid that is suitable for solution in, or suspension in, liquid prior to injection. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

Oral formulations will be in the form of solids or liquids, and may be solutions, syrups, suspensions, tablets, pills, capsules, sustained-release formulations, or powders. Oral formulations include such normally employed excipients as, for example, pharmaceutical grades of mannitol, lactose, starch, magnesium stearate, sodium saccharin, cellulose, magnesium carbonate, and the like.

Topical formulations will generally take the form of suppositories, pessaries, intranasal sprays or aerosols, buccal or sublingual tablets or lozenges. For suppositories or pessaries, traditional binders and excipients may include, for example, polyalkylene glycols or triglycerides; such suppositories or pessaries may be formed from mixtures containing the active ingredient. Other topical formulations may take the form of a lotion, solution, cream, ointment or dusting powder that may optionally be in the form of a skin patch.

The amount of each agent of the combination of the invention which is administered will vary depending upon factors such as the specific nature of the agent used, the size and health of the patient, the nature of the condition being treated etc. in accordance with normal clinical practice. Typically, a dosage in the range of from 0.01-1000 mg/kg, for instance from 0.1-10 mg/kg, would produce a suitable protective or cytotoxic effect. In this case however, the dosage of the CDC7 inhibitor (i) is required only to be sufficient to arrest normal cells in the G1 phase. This dosage may be lower than that conventionally used when that agent is intended to act in a therapeutic context in its own right. For example, where the inhibitor is a CDC7 inhibitor, such as TAK-931, LY-3143921, LBS-007, SRA-141, NMS-1116354, BMS-863233, RXDX-103, MSK-777 or MSK-747, the dosage may be in the range of from 30 mg-100 mg/kg. Furthermore, the fact that the agent (i) provides protection for normal cells means that the cytoxic agent (ii) may be better tolerated by the patient and therefore dosages at the higher end of the allowable range may be utilised. For example, where the cytoxic agent (ii) is paclitaxel, a dosage in the range of from 100-175 mg/square meter may be used, and wherein the cytotoxic agent is 5-fluorouracil, a dosage in the range of from 400-600 mg/sq. meter may be used.

Dosages may be given in a single dose regimen, split dose regimens and/or in multiple dose regimens lasting over several days. Effective daily doses will, however vary depending upon the inherent activity of the therapeutic agent, such variations being within the skill and judgment of the physician.

The combination of the present invention may be used in further combination with one or more other active agents, such as one or more pharmaceutically active agents and in particular other anti-cancer drugs, such as anti-hormonal agents. These can be administered at a time deemed suitable by a physician.

In a further aspect the invention provides a method of treating cancer, said method comprising administering to a patient in need thereof, a combination as described above. Suitable dosage regimes are also described above.

In the cyclotherapy study reported herein, the applicants sought to determine whether the origin activation checkpoint can be exploited to protect normal proliferating cells from cell-cycle-phase-specific chemotherapy. The potent anti-mitotic agent, paclitaxel was selected as a cytotoxin as cytotoxicity mediated by M-phase agents is strictly dependent on the cell's ability to enter mitosis. Furthermore, mitotic inhibitors including taxanes and Vinca alkaloids are in clinical utilization for a broad range of solid and hematological cancers, including metastatic and refractory disease. However, despite these clinical successes, severe side effects such as neurotoxicity, and the development of resistance can limit the utility of these agents. Therefore, identifying a basis for selective tumor cell killing yet sparing normal cycling cells could significantly enhance the therapeutic potential of these anti-mitotic agents.

The applicants have found that the DNA origin activation checkpoint actively prevents progression through a lethal S phase in primary epithelial and mesenchymal cells in the absence of a sufficient number of replication competent origins. This checkpoint is dependent on p53 function, in keeping with earlier observations. Inactivation of p53 in primary cells is sufficient to impair the origin activation checkpoint resulting in an abortive S phase and apoptotic cell death. It is well known that most solid tumor types are associated with p53 inactivation, and hence all four p53-deficient tumor cell lines included in this study exhibited an impaired checkpoint response when challenged with Cdc7 RNAi, leading to an abortive S phase and apoptosis. Without being bound by theory, this may be attributable not only to the critical role Cdc7 kinase plays in DNA replication initiation, but also to its reported roles in the DNA damage response (Montagnoli A. et al. Clin Cancer Res 2010; 16:4503-8). Under Cdc7 rate limiting conditions, cancer cells fail to arrest and progress into S phase despite the presence of an insufficient numbers of replication competent origins, most likely due to aberrations in TSGs involved in the origin activation checkpoint response, which can lead to fork stalling/collapse, DNA strand breaks and apoptotic cell death. Under normal conditions, fork stalling leads to origin firing from dormant origins which promotes completion of replication and cell survival, however, loss of Cdc7 activity would circumvent this failsafe mechanism.

Cdc7 has also been reported to be involved in activation of the DNA damage ATR-Chk1 checkpoint pathway though direct phosphorylation of claspin. Fork stalling can lead to activation of this checkpoint, therefore loss of Cdc7 activity uncouples ATR from Chk1 mediated cell cycle arrest and DNA repair, and lead to ATR dependent activation of p38 MAP kinase and apoptotic cell death.

Finally, Cdc7 was recently implicated to play a role in trans-lesion synthesis (TLS), via phosphorylation of Rad18 which is required for recruitment of TLS polymerases to stalled replication forks. TLS is a mode of DNA damage tolerance which maintains replication fork progression on damaged DNA, therefore loss of Cdc7 activity would result in impaired TLS and tolerance to DNA damage, fork stalling/collapse and cell death.

Most importantly, the applicants have demonstrated that by exploiting the differential checkpoint response to Cdc7 RNAi between normal and cancer cells, sequential treatment with Cdc7 RNAi and paclitaxel leads to synergistic cancer-cell-specific killing, while the arrested primary epithelial and mesenchymal cells remain completely shielded from paclitaxel cytotoxicity.

This therefore provides a novel strategy to enhance the therapeutic window of mitotic inhibitors. The exact mechanism of how Cdc7 RNAi potentiates paclitaxel mediated cancer cell killing is unclear, however it is likely that the proportion of cells which manage to escape an abortive S phase due to Cdc7 depletion are subsequently killed in M phase by a cytotoxin such as paclitaxel.

In addition, Cdc7 depletion may also sensitize cancer cells to cytotoxins such as paclitaxel. In the present study we show the applicability of this strategy in p53-deficient tumors, however due to the reliance of the origin activation checkpoint on several TSGs, it is also likely to be applicable to tumors with aberrations in other TSGs involved in the checkpoint response.

Several companies are developing low nanomolar Cdc7 inhibitors, but there are limited data available on these compounds and available Cdc7 inhibitors have been shown to exhibit cross-reactivity and so in this study we chose to inhibit Cdc7 using RNAi. However, the data provides support for combinations of Cdc7 inhibitors and cytotoxins as potent anti-cancer treatments in a wide range of solid tumor types. Notably Cdc7 lies at the convergence point of upstream oncogenic growth signalling pathways. Targeting Cdc7 may therefore potentially overcome problems associated with pathway redundancy and cancer cell cycles that have become growth independent (so-called autonomous cancer cell cycles).

Thus, the applicants have demonstrated that combining inhibitor of Cdc7 with mitotic inhibitors provides a useful cyclotherapeutic approach to significantly lower the toxicity associated with chemotherapeutics and in particular this class of cell-cycle-phase specific chemotherapeutics in normal proliferating cells while enhancing cancer-cell-specific killing. The origin activation checkpoint arrest in normal cells provides cellular protection for those self-renewing tissues with high turnover, and thereby circumvent the associated toxic side effects. Although the applicants have specifically used Cdc7 within the following experimental protocol it is established in the art that as discussed above other inhibitors or disruptors of the DNA licensing machinery and DNA replication initiation machinery exist and that a person skilled in the art would know that they would function as described herein.

Such a treatment regimen may allow increased dosage and frequency of cell-cycle-phase-specific agents, thus increasing the therapeutic window and reducing the chance for emergence of drug resistant clones.

### DETAILED DESCRIPTION OF THE INVENTION

The invention will now be particularly described by way of example with reference to the accompanying diagrammatic drawings in which:

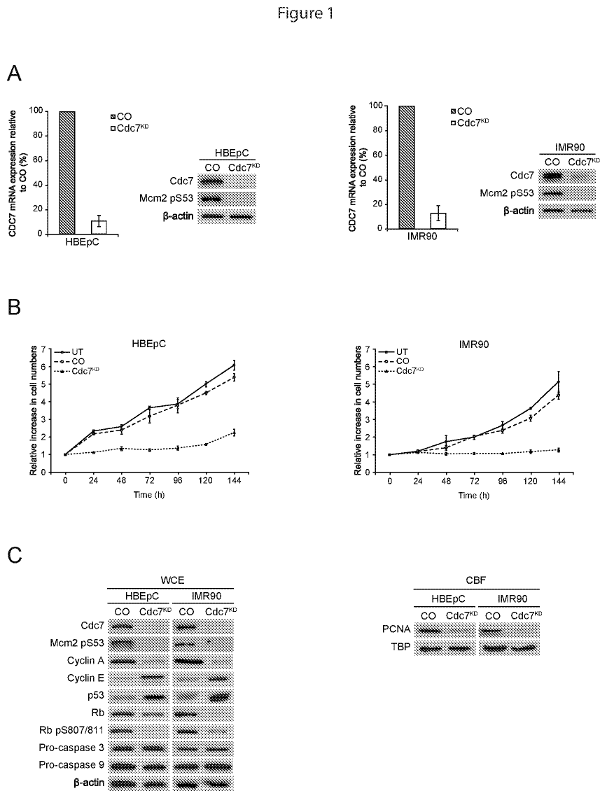

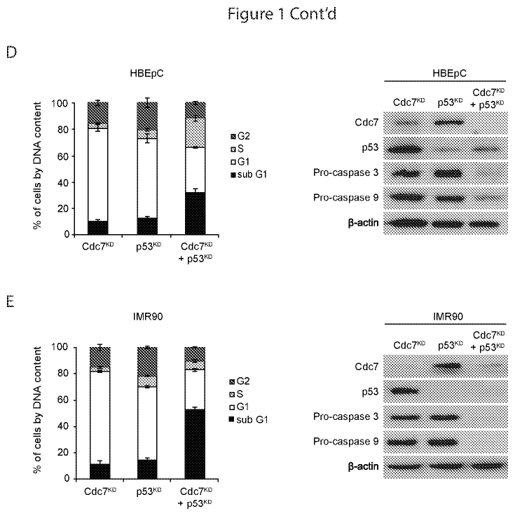

FIG. 1: illustrates that Cdc7 depletion in primary human epithelial and mesenchymal cells induces cell cycle arrest, and the impact of p53 on this. (A) Left panels: The expression of Cdc7 mRNA in CDC7-siRNA transfected (Cdc7KD) HBEpC and IMR90 cells relative to control-siRNA transfected (CO) at 72 hours post-transfection. Right panels: Immunoblot analysis of whole cell extracts (WCE) prepared from control-siRNA (CO) and CDC7-siRNA (Cdc7KD) transfected HBEpC and IMR90 cells 72 hours post-transfection with the indicated antibodies (_-actin—loading control). (B) Relative increase in cell numbers in Cdc7-depleted (Cdc7KD) HBEpC and IMR90 cells compared with untreated (UT) and control-siRNA transfected (CO) cells over a 144 hour time course. Error bars represent standard error of the mean for three experiments. (C) Immunoblot analysis of WCE and chromatin-bound protein fractions (CBF) prepared from control-siRNA (CO) and CDC7-siRNA (Cdc7KD) transfected HBEpC and IMR90 cells 72 hours post-transfection with the indicated antibodies (_-actin and TBP—loading controls). (D) Cell viability and immunoblot analysis of doubly-transfected HBEpC and (E) IMR90 cells, transfected with control-siRNA (CO) or CDC7-siRNA (Cdc7KD) for 48 hours followed by transfection with CDC7-siRNA or p53-siRNA (p53KD) for a further 72 hours. Bar charts show the cell cycle distribution as monitored by flow cytometry. Error bars represent standard error of the mean for three experiments. Right panels show immunoblots of WCE prepared from siRNA-transfected HBEpC and IMR90 cells probed with the indicated antibodies (_-actin—loading control).

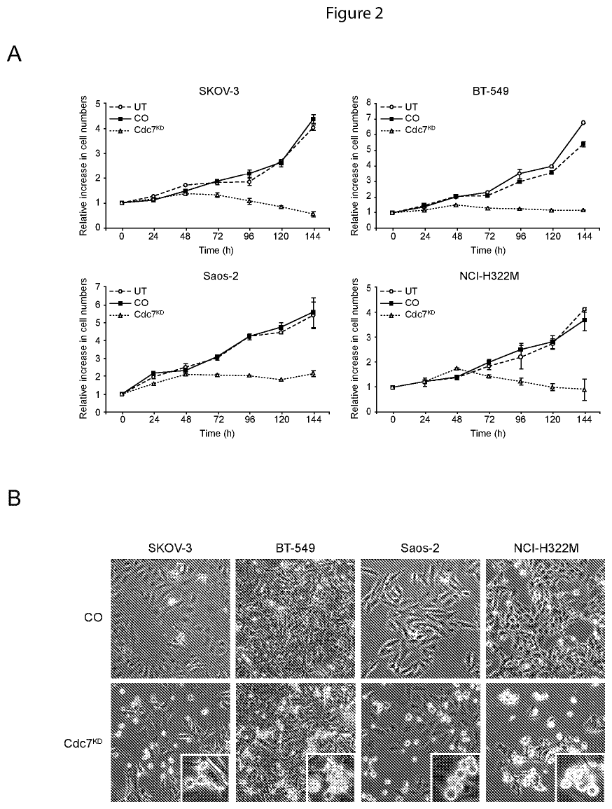

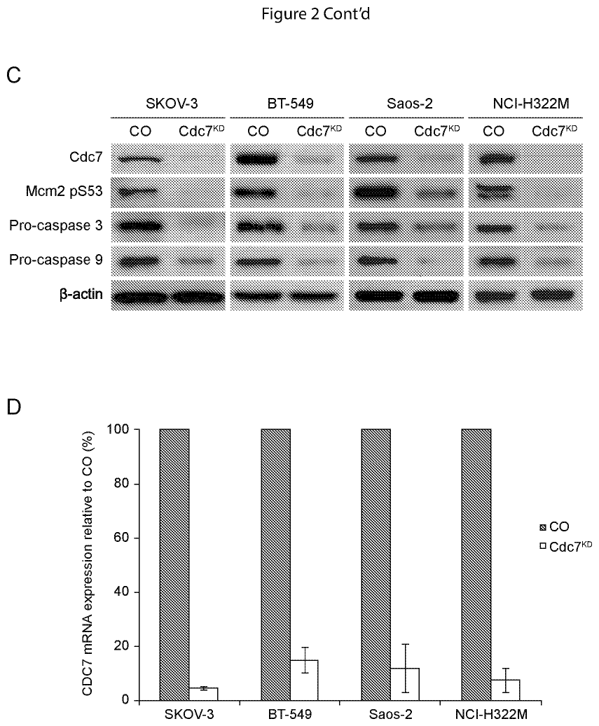

FIG. 2: Cdc7 loss induces apoptosis in p53-deficient cancer cell lines. (A) Relative increase in cell numbers in Cdc7-depleted (Cdc7KD) SKOV-3, BT-549, Saos-2 and NCIH332M cancer cells compared to untreated (UT) and control-siRNA transfected (CO) cells over a 144 hour time course. Error bars represent standard error of the mean for three experiments. (B) Phase contrast microscopy images of SKOV-3, BT-549, Saos-2 and NCIH332M cancer cells 96 hour post-transfection. (C) Immunoblot analysis of WCE prepared from control-siRNA (CO) and CDC7-siRNA (Cdc7KD) transfected SKOV-3, BT-549, Saos-2 and NCI-H332M cancer cells 96 hours post-transfection with the indicated antibodies (_-actin—loading control). (D) efficiency of Cdc7 knockdown in cancer cells. CDC7 Mrna expression in Cdc7-depleted (Cdc7KD) SKOV-3, BT-549, Saos-2 and NCI-H332M cancer cells relative to control-siRNA (CO) transfected cells 96 hours post-transfection.

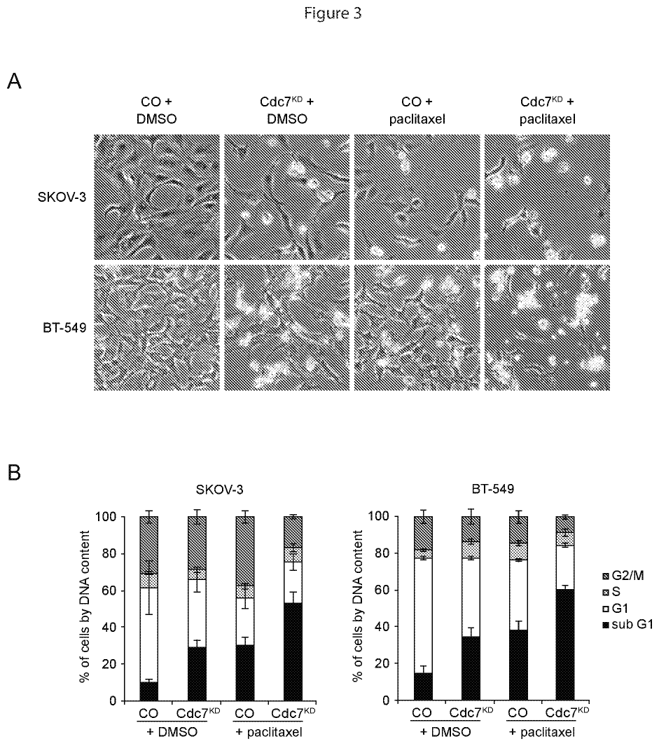

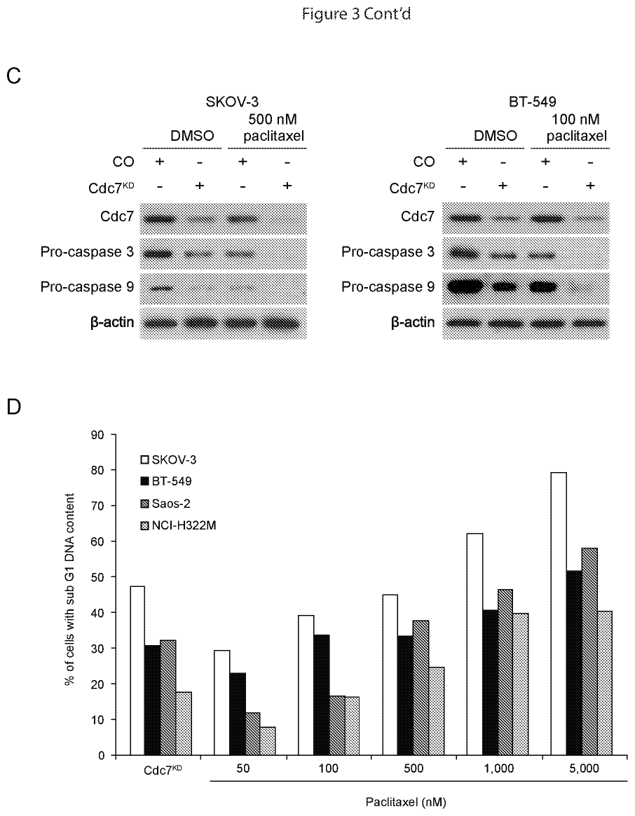

FIG. 3: Cdc7 loss enhances paclitaxel cytotoxicity in cancer cells. SKOV-3 and BT-549 cancer cells were cultured for 24 hours with 500 nM and 100 nM paclitaxel, respectively, or DMSO following pre-treatment, 48 hours earlier, with CDC7-siRNA (Cdc7KD) or control-siRNA (CO). Apoptosis was monitored by (A) phase contrast microscopy, (B) flow cytometric detection of cells with sub-G1 DNA content, and (C) western blot detection of pro-caspase 3 and pro-caspase 9 cleavage in WCE. Error bars represent standard error of the mean for three experiments. (D) Concentrations of paclitaxel optimized to induce apoptosis comparable to Cdc7 depletion in cancer cells. Percentage of SKOV-3, BT-549, Saos-2 and NCI-H322M cancer cells with sub-G1 DNA content following Cdc7 depletion for 72 hours (Cdc7KD) or with increasing concentrations of paclitaxel for 24 hours.

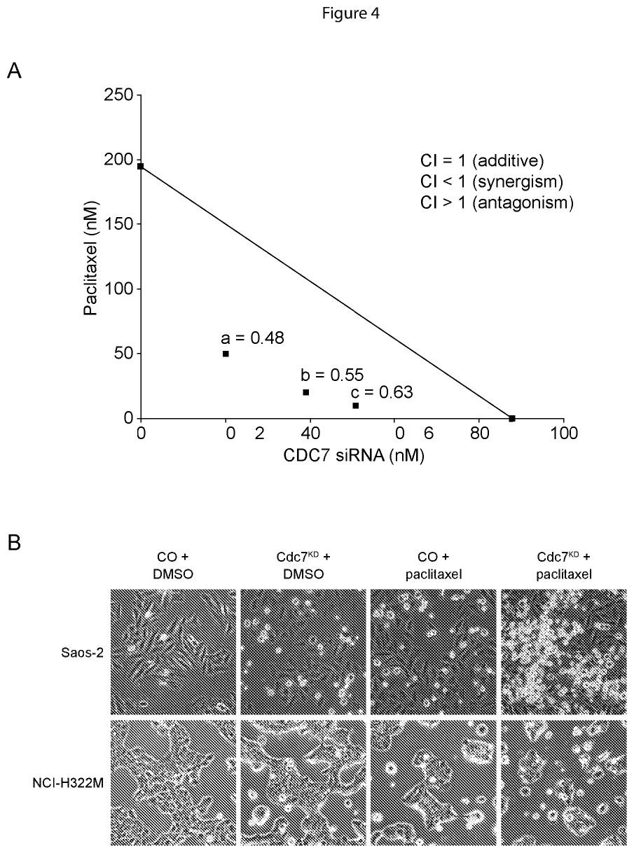

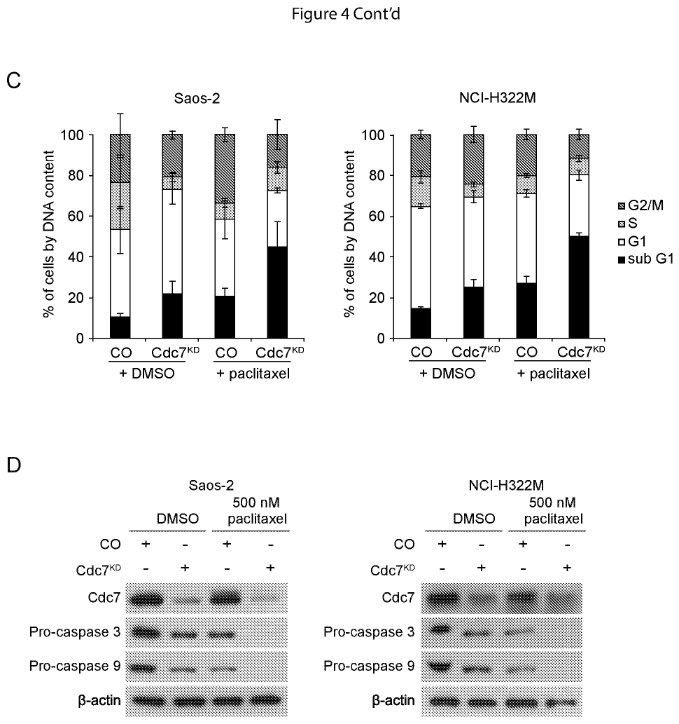

FIG. 4: Cdc7 depletion interacts synergistically with paclitaxel in cancer-cell-specific killing. (A) Classic ED50 isobologram depicting the interaction between Cdc7 knockdown and paclitaxel in BT-549 cells. The ED50 values of different equipotent combinations of Cdc7-siRNA and paclitaxel are plotted on the graph as data points a, b and c. The combination indexes of data points a, b and c are as indicated. Cdc7 loss enhances paclitaxel cytotoxicity in cancer cells. Saos-2 and NCI-H322M cancer cells were cultured for 24 hours following addition of 500 nM paclitaxel or DMSO following pre-treatment, 48 hours earlier, with CDC7-siRNA (Cdc7KD) or control-siRNA (CO). Apoptosis was monitored by (B) phase contrast microscopy, (C) flow cytometric detection of cells with sub-G1 DNA content, and (D) western blot detection of pro-caspase 3 and procaspase 9 cleavage in WCE. Error bars represent standard error of the mean for three experiments.

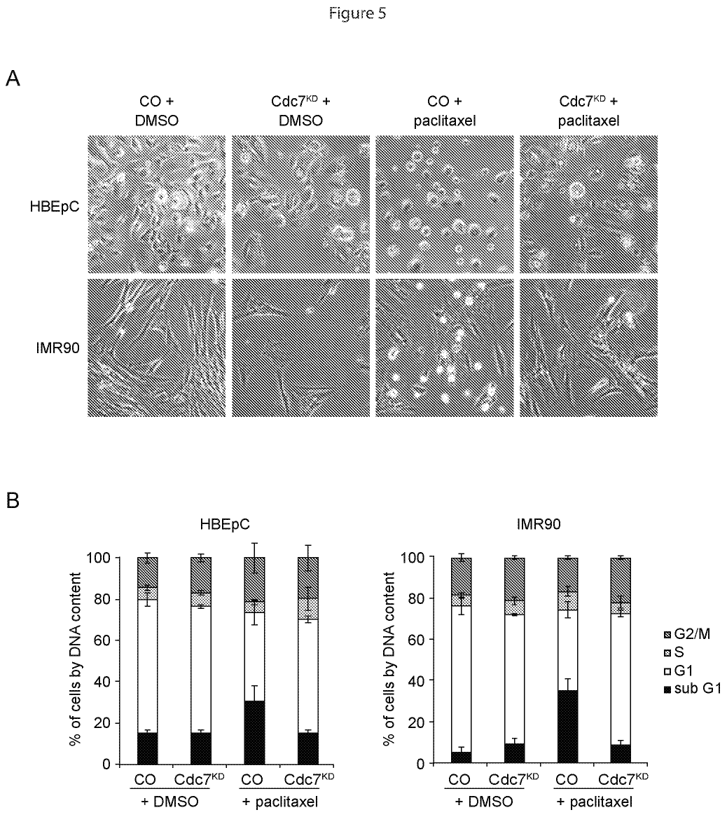

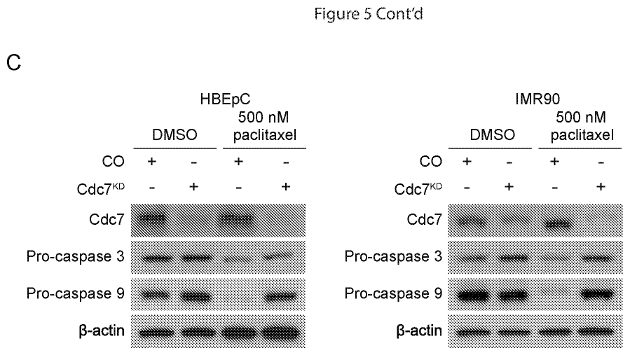

FIG. 5: Activation of the DNA replication origin activation checkpoint shields primary cells from paclitaxel cytotoxicity. HBEpC and IMR90 primary cells were cultured for 24 hours with 500 nM paclitaxel or DMSO following pre-treatment, 48 hours earlier, with CDC7-siRNA (Cdc7KD) or control-siRNA (CO). Apoptosis was monitored by (A) phase contrast microscopy, (B) flow cytometric detection of cells with sub-G1 DNA content, and (C) western blot detection of pro-caspase 3 and pro-caspase 9 cleavage in WCE. Error bars represent standard error of the mean for three experiments.

FIG. 6: is a schematic representation of the activity of the combination of the invention on an untransformed (normal) cell and a cancer cell.

### EXAMPLE 1

CDC7 Knockdown Causes Cell Cycle Arrest in Primary Human Mesenchymal and Epithelial Cells

As discussed above, it has been reported that Cdc7 depletion in primary cells leads to a G1 cell cycle arrest, pointing towards an origin activation checkpoint. This was investigated in two different primary untransformed cell types to ensure that this checkpoint is conserved.

IMR90 mesenchymal diploid fibroblasts (CCL-186, ATCC) were cultured in Dulbecco's Modified Eagle's medium (31966, Invitrogen) supplemented with 10% Fetal Bovine Serum (FBS) (10270-106, Invitrogen). HBEpC bronchial epithelial primary cells (ECACC, 502-05a) were cultured in Bronchial Epithelial Cell Serum Free growth medium (Ser. No. 06/091,518, ECACC).

Cells were then transfected with CDC7-siRNA to inhibit CDC7 expression. Specifically, CDC7 expression was inhibited with custom double-stranded RNA oligos (5′-GCUCAGCAGGAAAGGUGUUUU-3′ (SEQ ID NO 1) and 5′-AACACCUUUCCUGCUGAGCUU-3′ (SEQ ID NO 2) Thermo Scientific), using previously described methods (Tudzarove S. et al 2010 supra.). Non-targeting siRNA was used as a negative control (12935-300, Invitrogen). CDC7 or control siRNA duplexes were transfected using Lipofectamine 2000 (11668019, Invitrogen) according to the manufacturer's recommendations at an optimized concentrations of 10 nM for IMR90 and 25 nM for HBEpC.

At 72 hours post-transfection, CDC7 mRNA levels were measured using qPCR in order to evaluate the efficiency of transfection with CDC7. In particular, Total RNA was isolated from cells with the PureLink Microto-Midi Total RNA Purification System (Invitrogen) according to the manufacturer's instructions. Total RNA (40 ng) was reverse transcribed, and real-time PCR was performed using a SuperScript III Platinum SYBR Green OneStep qRTPCR kit (Invitrogen) following the manufacturer's instructions. Reactions were carried out in an Eppendorf Mastercycler ep Realplex Real-Time PCR System (Eppendorf, Cambridge, UK), and results were analyzed with Realplex v1.5 software (Eppendorf). Primer sequences were: CDC7 forward 5′-AACTTGCAGGTGTTAAAAAAG-3′ (SEQ ID NO 3) and reverse 5′-TGAAAGTGCCTTCTCCAAT-3′ (SEQ ID NO 4); GAPDH (invariant control) forward 5′-TCAACTACATGGTTTACATGTTC-3′ (SEQ ID NO 5) and reverse 5′-GATCTCGCTCCTGGAAGAT-3′ (SEQ ID NO 6).

The qRT-PCR protocol was as follows: a single cDNA synthesis step case was performed at 50° C. for 3 minutes; followed by a single denaturation step at 95° C. for 10 minutes; then 45 cycles of denaturation at 95° C. for 15 seconds, annealing at 47° C. for 20 seconds and extension at 60° C. for 20 seconds. The Eppendorf Realplex Detection System Software (Eppendorf, Hamburg, Germany) was used to determine Cycle threshold (Ct) values. GAPDH measurements were used to normalize the data and the www.impactjournals.com/oncotarget 18505 Oncotarget relative expression of CDC7 RNA in treated samples to untreated samples was determined The results are shown in FIG. 1A. CDC7 mRNA levels were reduced by 89% and 87% in CDC7-siRNA (Cdc7KD) transfected HBEpC and IMR90 cells, respectively, relative to control siRNA transfected (CO) cells.

In addition, whole cell extracts (WCE) were prepared by cell lysis in modified RIPA buffer (50 mmol/L Tris-Cl pH 7.4, 300 mmol/L NaCl, 0.1% NP40, 1% Triton X-100, 0.5% sodium deoxycholate, 0.1% SDS, 5 mmol/L EDTA, 1 mmol/L EGTA, 100 mmol/L sodium fluoride and 1 mmol/L sodium orthovanadate) followed by sonication for 10 seconds. These were analysed by Western Blotting, in which fifty micrograms of protein was loaded in each lane, separated by 4-20% SDS-PAGE, and transferred by semidry electroblotting onto Hybond C Extra nitrocellulose membranes (GE Healthcare, Buckinghamshire, UK).

Primary antibodies used were: caspase 3 (NB500-210, Novus Biologicals); caspase 9 (sc-17784, Santa Cruz Biotechnology); Cdc7 (K0070-3, MBL International); cyclin A (sc-596, Santa Cruz Biotechnology); cyclin E (MS-870-P, NeoMarkers); Mcm2 pS53 (A300-756A, Bethyl Laboratories); PCNA (610665, BD Biosciences); Rb (554136, BD Biosciences); Rb pS807/811 (9308, Cell Signaling Technologies); TBP (A301-229A, Bethyl Laboratories).

Blocking, antibody incubations (using various antibodies including anti-Cdc7 or anti-Mcm2 pS53 antibodies), and washing steps were performed.

The results, provided in FIG. 1A, show that CDC7 protein levels were undetectable in both cell lines. Furthermore, consistent with efficient Cdc7 depletion 72 hours post-transfection, Mcm2 phosphorylation at Ser-53 was abolished in both cell lines (FIG. 1A).

Phase contrast microscopy was performed over a period of 144 hours with an inverted Aziover 200m (Carl Zeiss, Welwyn Garden City, Uk) and Axiovision software to assess cell population growth. The results are shown in FIG. 1B. CDC7 downregulation resulted in cessation of cell proliferation in both lines, with cell numbers reaching a plateau 48 hours post-transfection until 120 hours post-transfection.

In line with this, flow cytometry analysis of Cdc7-depleted HBEpC and IMR90 cells showed an accumulation consistent with a G1 arrest 72 hours post-transfection.

Additional western blotting of WCE prepared from HBEpC and IMR90 cells showed that Cdc7-depletion led to an increase in cyclin E levels, a reduction in S phase cyclin A, loss of Rb phosphorylation at Ser-807/811, thought to be either Cdk4 or Cdk2 phosphorylation sites, 21, 22 and p53 stabilization (FIG. 1C).

In addition chromatin-bound levels of DNA polymerase progressivity factor PCNA were determined (S. R. Kingsbury et al. Experimental Cell Research 309 (2005) 56-67 57). The total protein content into cytosolic, nucleosolic and chromatin-bound fractions was separated by pelleting cells, resuspending pelleted cells in buffer A (10 mM HEPES, pH 7.9, 10 mM KCl, 1.5 mM MgCl2, 0.34 M sucrose, 10% glycerol, 1 mM DTT, 5 Ag/ml aprotinin, 5 Ag/ml leupeptin, 0.5 Ag/ml pepstatin A, 0.1 mM PMSF) at 4107 cells/ml and incubating for 10 minutes on ice. Nuclei were pelleted by low speed centrifugation (1300 g, 5 min, 4° C.), and the supernatant (cytosolic fraction) further clarified by high speed centrifugation (14,000 g, 15 min, 4-C). Nuclei were washed twice in buffer A, resuspended in 3 mM EDTA, 0.2 mM EGTA, 1 mM DTT, 5 Ag/ml aprotinin, 5 Ag/ml leupeptin, 0.5 Ag/ml pepstatin A and 0.1 mM PMSF and incubated for 45 min at 4° C. Insoluble chromatin-bound proteins were obtained by centrifugation (1500 g, 5 min, 4° C.), and the supernatant (nucleosolic fraction) further clarified by high speed centrifugation (14,000 g, 15 min, 4-C). Chromatin was washed twice, resuspended in buffer A plus 1000 U/ml DNase I (Invitrogen, Paisley, UK) and incubated for 30 min at RT and a further 30 min at 4° C. with 1 volume 0.5 M NaCl. Solubilised chromatin-bound proteins were obtained by high speed centrifugation (14,000 g, 5 min, 4° C.).

It was found that chromatin-bound levels of the DNA polymerase processivity factor PCNA were markedly reduced in Cdc7-depleted cells (FIG. 1C). This cyclin profile, low CDK activity and loss of chromatin-bound PCNA is therefore consistent with the observed cessation of cell proliferation (FIG. 1B) and a G1 cell cycle arrest.

Moreover, phosphorylation of p53 at SE-15 and Chk1 at Ser-345 were not detected (FIG. 1C), suggesting that the Cdc7-depletion induced cell cycle arrest was not triggered by the ATM/ATR DNA damage repair pathways (p53-Ser15) or in response to blocked DNA replication (Chk1-Ser345). 23, 24 Additionally, Cdc7-depleted cells with less than 2C (sub G1) DNA content were not detected by flow cytometry, nor was cleavage of pro-caspases 3 and 9 (FIG. 1C), indicating that the arrested cells did not activate the cell death effector machinery and remained viable. The above data is in line with previous work, and confirms that Cdc7-depletion in primary cells of epithelial and mesenchymal origin induces a cell cycle arrest, and is not associated with loss of cell viability.

### EXAMPLE 2

Loss of Function of p53 Disables the Origin Activation Checkpoint in Transformed Cells

A dependency on p53 for Cdc7-depletion induced cell cycle arrest has been shown for human dermal and lung fibroblasts, and mammary epithelial cells. The present observation that p53 is also stabilized in bronchial epithelial cells arrested by Cdc7 depletion (FIG. 1C) is consistent with an active role for p53 in the underlying cell cycle checkpoint that is conserved between different primary cell lines of mesenchymal and epithelial origin. To confirm the p53 dependency of this checkpoint in both the primary cell lines used in the present study, RNAi was used to inhibit p53 expression in HBEpC and IMR90 cells previously arrested by Cdc7 depletion.

For double-transfection with CDC7 and p53 siRNAs (p53 specific duplex, sense 5′-GGA AGA CUC CAG UGG UAA UUU-3′ (SEQ ID NO 7) and antisense 5′-AUU ACC ACU GGA GUC UUC CUU-3′ (SEQ ID NO 8) and ON-TARGETplus SMARTpool TP53 L-003329-00 [Thermo Scientific]), HMEpC and MCF10A cells were first transfected with 10 nmol/L CDC7 or control (Luciferase siRNA, Ambion, Austin, Tex.) siRNA. After 24 hours medium was removed and cells were retransfected with control (20 nmol/L), CDC7 (10 nmol/L) plus control (10 nmol/L), or CDC7 (10 nmol/L) plus p53 (9 nmol/L duplex plus 1 nmol/L SMARTpool) siRNA mixtures. Cells were harvested at the indicated time points after the second transfection. Efficient knockdown was assessed by qRT-PCR and/or Western blot.

Flow cytometry of doubly-depleted Cdc7/p53 cells (FIGS. 1D and 1E, left panels) showed a marked reduction in the percentage of cells with a G1 DNA content and a concomitant increase in the number of cells with S phase DNA content. Moreover, a 3-fold and 5-fold increase in cells with less than 2C (sub G1) DNA content was observed for doubly-depleted HBEpC and IMR90 cells, respectively, compared to cells depleted of either Cdc7 or p53 alone (FIGS. 1D and 1E, left panels).

Consistent with this, cleavage of pro-caspases 3 and 9 (FIGS. 1D and 1E, right panels) confirmed the induction of apoptosis in doubly-depleted Cdc7/p53 cells. In line with previous studies, the results show that loss of p53 in primary cells disables the origin activation checkpoint, allowing cells to bypass the cell cycle arrest leading to an abortive S phase followed by apoptosis and therefore support the emerging concept in cancer therapy of targeting DNA replication before it starts.

On this basis, RNAi was then used to investigate the effects of Cdc7 loss in epithelial and mesenchymal cancer cell lines deficient in functional p53. In this experiment, SKOV-3 ovarian carcinoma cells (HTB-77, ATCC) and Saos-2 osteosarcoma cells (HTB-85, ATCC) in McCoy's 5A medium (26600, Invitrogen) supplemented with 15% FBS, BT-549 mammary ductal carcinoma cells (HTB-122, ATCC) in RPMI-1640 medium (52400, Invitrogen) supplemented with 10% FBS and 10 μg/ml insulin (10516, Sigma); and NCI-H322M bronchioalveolar carcinoma cells (95111734, ECACC) in RPMI-1640 medium were used.

Cells were transfected with siRNA as described in Example 1 and the effects on mRNA expression, western blotting and cell population growth were assessed also as described in Example 1.

CDC7 knockdown in p53 null SKOV-3 ovarian carcinoma and Saos-2 osteosarcoma cells, and p53 mutant BT-549 breast carcinoma and NCI-H322M small cell lung carcinoma cells was confirmed by qPCR (FIG. 2D). Furthermore, CDC7 depletion caused a cessation of cell proliferation, with cell numbers reduced between 3- and 7-fold compared to control cells 144 hours post-transfection (FIG. 2A). Induction of apoptosis in Cdc7-depleted cancer cells lacking functional p53, but not in cells transfected with control-siRNA, was confirmed morphologically (FIG. 2B) and through western blot detection of cleavage of pro-caspases 3 and 9 in WCE 96 hours post-transfection (FIG. 2C). These results further support an essential role for p53 in the origin activation checkpoint, and suggest that p53 loss or inactivation during tumorigenesis contributes to the cancer-cell-specific killing reported for pharmacological Cdc7 inhibitors.

### EXAMPLE 3

CDC7 Knockdown Enhances Paclitaxel Toxicity in Transformed Cells

Since CDC7 depletion causes an abortive S phase and apoptotic cell death in cancer cells (FIG. 2), the applicants postulated that pharmacological Cdc7 inhibitors could be used not only as a single agent therapeutic but also in combination with existing chemotherapy, such as the potent anti-mitotic paclitaxel. We therefore tested the hypothesis that CDC7 knockdown in combination with paclitaxel would increase cancer cell killing compared with single agent treatment.

SKOV-3, BT-549, Saos-2 and NCI-H322M cells as described in Example 2 were cultured for 24 hours with increasing paclitaxel concentrations (50 nM-5 mM) to determine a concentration that resulted in a similar level of cancer cell killing compared to treatment with CDC7-siRNA after 72 hours (FIG. 3D). Next, SKOV-3, BT-549, Saos-2 and NCI-H322M cells were transfected with CDC7-siRNA or control-siRNA 48 hours prior to treatment with 100 nM (BT-549) and 500 nM (SKOV-3, Saos-2, NCI-H322M) paclitaxel or DMSO and the cells cultures for a further 24 hours.

Phase contrast microscopy revealed the presence of apoptotic cells in all four cancer cell lines receiving single agent treatment with either CDC7-siRNA or paclitaxel.

As predicted, an increase in the number of apoptotic cells was observed in cells receiving the combination treatment of CDC7-siRNA and paclitaxel (FIG. 3A and FIG. 4B). Flow cytometry confirmed an approximately 2-fold increase in the percentage of cells with less than 2C (sub G1) DNA content for SKOV-3, BT-549, Saos-2 and NCI-H322M cells receiving the combination treatment compared with single agent CDC7-siRNA or paclitaxel treatment (FIG. 3B and FIG. 4C). Consistent with this, all four cancer cell lines showed a marked increase in pro-caspase 3 and 9 cleavage after the combination treatment compared with single agent treatment (FIG. 3C and FIG. 4D).

Finally, to determine whether the increased killing observed in cancer cells receiving combination treatment is due to an additive or synergistic interaction between CDC7-siRNA and paclitaxel, isobologram and CI analyses were performed in the BT-549 cells, as a representative p53-deficient cancer cell line.

Cell viability was determined using the XTT assay (11465015001, Roche) according to the manufacturer's instructions. The concentrations of CDC7-siRNA and paclitaxel used alone and in combination to reduce cell viability by 50% (i.e. ED50) were determined. For single agent treatment cell viability assays, cells were treated with CDC7-siRNA and cultured for 72 hours or treated with paclitaxel and cultured for 24 hours. For combination treatments, cells were treated with CDC7-siRNA 48 hours prior to treatment with paclitaxel and then cultured for a further 24 h. Experiments were performed in triplicate. Dose-response curves and ED50 values were generated using Prism version 4.0 (Graphpad Software, Inc.). A classic ED50 isobologram was produced using Microsoft Excel by plotting ED50 CDC7-siRNA on the x17 axis and ED50 paclitaxel on the y-axis. A diagonal line was used to connect ED50 CDC7-siRNA and ED50 paclitaxel and represents the line of additivity. Experimentally derived equipotent combinations of CDC7-siRNA and paclitaxel (ED50 CDC7-siRNA+paclitaxel) were then plotted on the isobologram. ED50 CDC7-siRNA+paclitaxel values which lie on, above or below the line of additivity indicate an additive, antagonistic or synergistic interaction between CDC7 knockdown and paclitaxel in BT-549 cells.

Combination indexes were determined using the CI equation method described by Chou and Talalay, 48 and the equation CI=[ED50 CDC7+Pac]/[ED50 CDC7]+[ED50 Pac+CDC7]/[ED50 Pac]. ED50 CDC7 and ED50 Pac are the ED50 of CDC7-siRNA and paclitaxel when used alone, respectively. ED50 CDC7+Pac and ED50 Pac+CDC7 are the ED50 of CDC7-siRNA and paclitaxel when used in combination, respectively. CI values=1, <1 or >1 indicate an additive, synergistic or antagonistic effect, respectively.

The results demonstrate that the experimentally derived equipotent combination treatment ED50 data points lie below the line of additivity, and have quantitative CI values<1, indicating that CDC7-siRNA acts synergistically with paclitaxel in relation to cancer cell killing (FIG. 4A). The above data therefore suggest that combination therapy with Cdc7 inhibitors and cytotoxic agents such as M-phase agents like paclitaxel could enhance cancer-cell specific killing.

### EXAMPLE 4

The Origin Activation Checkpoint Shields Primary Cells from Paclitaxel Toxicity

CDC7 depletion triggers the origin activation checkpoint mediated G1 cell cycle arrest in normal untransformed cells, but not in cancer cells. Therefore, together with the above data (FIGS. 3 and 4), the applicants postulated that by exploiting the differential checkpoint response between normal and cancer cells, sequential treatment with Cdc7 RNAi and paclitaxel can enhance cancer-cell-specific killing while shielding normal cycling cells from taxane associated toxicity, thereby increasing the therapeutic window.

To test this hypothesis, HBEpC bronchial epithelial cells and IMR90 mesenchymal fibroblasts were transfected with CDC7-siRNA or control-siRNA 48 hours prior to treatment with 500 nM paclitaxel or DMSO and then cultured for a further 24 hours. Cells were harvested and apoptosis was monitored by phase contrast microscopy, flow cytometry and western blot detection of pro-caspase 3 and 9 cleavage.

HBEpC and IMR90 cells transfected with control-siRNA and treated with paclitaxel showed morphological signs of apoptosis, while cells depleted of Cdc7 and treated with paclitaxel were morphologically indistinguishable from cells treated with DMSO alone (FIG. 5A). Flow cytometry revealed small proportions of cells with less than 2C DNA content amongst populations of HBEpC (15%) and IMR90 (4%) control-siRNA transfected cells treated with DMSO (FIG. 5B). As expected, the percentage of cells with less than 2C (sub G1) DNA content increased to 30% and 35% for HBEpC and IMR90 control-siRNA transfected cells treated with paclitaxel (FIG. 5B). Notably, pre-treatment with CDC7-siRNA shielded both primary cell lines from apoptotic cell death, with only 15% of HBEpC and 8% of IMR90 cells treated with paclitaxel showing less than 2C DNA content, comparable to control-siRNA transfected cells treated with DMSO (FIG. 5B). Moreover, whilst there was a marked reduction in pro-caspase 3 and 9 levels in control-siRNA transfected cells treated with paclitaxel, indicating activation of the cell death machinery, levels found in cells treated with CDC7-siRNA prior to paclitaxel treatment were comparable to those observed in control-siRNA transfected and DMSO-treated cells (FIG. 5C).

These results demonstrate that the origin activation checkpoint operating in primary epithelial and mesenchymal cells can be exploited to shield normal proliferating cells from the cytotoxic effects of cytotoxic agents, and in particular, M-phase specific agents, such as paclitaxel.

1. A combination treatment of a patient in need thereof, comprising:
administering a combination of i) an inhibition or disruption agent which inhibits or disrupts DNA licensing machinery and/or DNA replication initiation machinery and ii) a cytotoxic agent which acts in either G2, M and/or S phases of a cell cycle, thereby shielding normal cells during cancer treatment of the patient, wherein the inhibition or disruption agent is administered to the patient first in an amount sufficient to reversibly arrest normal cells in G1 phase, and the cytotoxic agent is administered at a subsequent time.

2. The combination treatment according to claim 1 wherein the inhibition or disruption agent inhibits or disrupts one or more of Cdc-7, ORC1-6, Cdc6, MCM2-7, Cdt1, Dbf4 Cdc45, GINS, Polε, Mcm10, Sid3, Sid5, Sid7, Sid2, Dpb11, Polα, Ctf4, PCNA, Pfs1, Pfs2 and Psf3.

3. The combination treatment according to claim 1 wherein the cytotoxic agent is paclitaxel or 5-fluorouracil.

4. The combination treatment according to claim 1 wherein the subsequent time following administration of agent (i) is in the range of from 1-60 hours later.

5. The combination treatment according to claim 4 wherein the subsequent time following administration of agent (i) is in the range from 12 to 48 hours later.

6. A packaged dosage unit, comprising:
a first pharmaceutical composition comprising an inhibition or disruption agent which inhibits or disrupts the DNA licensing machinery and/or the DNA replication initiation machinery, and a pharmaceutically acceptable carrier; and
a second pharmaceutical composition comprising a cytotoxic agent which acts in one or more of G2, M and/or S phases of a cell cycle, and a pharmaceutically acceptable carrier.

7. A method of shielding normal cells during cancer treatment, said method comprising administering to a patient in need thereof, an effective amount of a combination according to claim 1; wherein the inhibition or disruption agent which inhibits or disrupts the DNA licensing machinery and/or the DNA replication initiation machinery (i) is administered to the patient and the cytotoxic agent (ii) is administered subsequently.

8. The method according to claim 7 wherein the inhibition or disruption agent (i) is administered to the patient and the cytotoxic agent (ii) is administered from 1-60 hours later.

9. The method according to claim 8 wherein the inhibition or disruption agent (i) is administered to the patient and the cytotoxic agent (ii) is administered from 12-48 hours later.


SEQUENCE LISTING (6 sequences)

SEQ ID NO: 1 - RNA, 21 nt, Unknown
  custom CDC-7siRNA
gcucagcaggaaagguguuuu

SEQ ID NO: 2 - RNA, 21 nt, Unknown
  custom CDC7-siRNA
aacaccuuuccugcugagcuu

SEQ ID NO: 3 - DNA, 21 bp, Unknown
  CDC7 forward primer
aacttgcaggtgttaaaaaag

SEQ ID NO: 4 - DNA, 19 bp, Unknown
  CDC7 reverse primer
tgaaagtgccttctccaat

SEQ ID NO: 5 - DNA, 23 bp, Unknown
  GAPDH forward primer
tcaactacatggtttacatgttc

SEQ ID NO: 6 - DNA, 19 bp, Unknown
  GAPDH reverse primer
gatctcgctcctggaagat